(19) United States
(12) Patent Application Publication
(10) Pub. No.: US 2026/0345905 A1
(43) Pub. Date: Nov. 5, 2026

(54) QUANTITATIVE INJECTION PEN AND ITS METHOD OF USE

(71) Applicant: SHANGHAI INNOPAC MEDICAL TECHNOLGY CO., LTD., Shanghai (CN)

(72) Inventor: Rong Guo, Shanghai (CN)

(21) Appl. No.: 19/488,834

(22) PCT Filed: Jul. 29, 2024
(86) PCT No.: PCT/CN2024/108078

§ 371 Date: Nov. 30, 2025

(30) Foreign Application Priority Data
May 31, 2023 (CN) 202310634919.5

(51) Int. Cl. A61M 5/20; A61M 5/24; A61M 5/315

(52) U.S. Cl. CPC A61M 5/20; A61M 5/24; A61M 5/31528

(57) ABSTRACT

The present invention pertains to the technical field of medical devices, and more particularly, to drug delivery apparatuses for precise infusion and quantitative injection of liquid medications, with specific relevance to a quantitative injection pen and its method of use. The quantitative injection pen comprises a lower housing accommodating a cartridge containing a medicament and configured for mounting an injection needle, a push rod having a distal end for urging a piston within the cartridge, a cycle wheel engaged with the push rod, an upper housing engaged with the cycle wheel, and a reset rod engaged with the upper housing. The reset rod is rotatable in a reset direction to a pre-injection angle to switch to an injection state, and subsequently rotatable in an injection direction to quantitatively administer the medicament. A quantitative system is provided within the injection pen to ensure delivery of a specific dosage per injection. Notably, when the reset rod is not fully rotated to the pre-injection angle, it can be reversely rotated back in the injection direction to a stored state without medicament injection, thereby providing an opportunity to abort the current injection procedure.

### TECHNICAL FIELD

The present invention belongs to the technical field of medical devices, and more specifically, to the technology of drug delivery equipment for achieving precise infusion and quantitative injection of liquid medications, with particular relevance to a quantitative injection pen and its method of use.

### BACKGROUND ART

A quantitative injection pen is a medical device designed for the precise administration of liquid medication. It typically consists of components such as a syringe, a needle, and a piston. Quantitative injection pens can help reduce potential injection errors, thereby improving dosing accuracy and safety. For instance, for osteoporosis patients, such pens can be used to administer medication and help manage the condition. Specifically, based on a doctor's prescription, a pre-filled syringe or vial containing an anti-osteoporosis drug, such as Teriparatide, is selected and loaded into the pen's cartridge. The doctor then strictly calculates and controls the dosage according to the patient's physique and condition, typically around 20 micrograms per injection. The injection procedure involves inserting the pen's needle into the patient's subcutaneous tissue or muscle and pressing the injection button to slowly deliver the drug. This button-press injection method is inherently designed to prevent overdosing by incorporating a maximum injection volume limit; the injection automatically stops when this preset limit is reached, thus avoiding overdose caused by operational errors.

In the prior art, for example, CN106999664B discloses a quantitative injection pen comprising: a housing; a dosing element movable relative to the housing between a start position and a dosed position; a drive system coupled to the dosing element and operable to initiate or perform a dispensing operation; and a sub-dose prevention system. This sub-dose prevention system prevents or blocks the dispensing operation of the drive system when the dosing element has not reached the dosed position, and allows or unlocks it when the dosing element is at or past the dosed position. When the dispensing operation is prevented or blocked, the sub-dose prevention system is operable to block rotational movement of a drive member of the drive system in a circumferential direction corresponding to the dispensing or dial-down direction. Specifically, this is achieved via a locking ring, which allows dial-up rotation or movement of a rotating sleeve when the preset dose has not been fully dialed up, but prevents its dial-down rotation or movement.

However, a drawback of the sub-dose prevention system in the aforementioned application is that if an operator intends to abort the current injection process before the dosing element reaches the dosed position, it cannot be achieved. Therefore, there is a current need for a quantitative injection pen that allows for reversal when the dosed position is not fully reached, while still effectively providing sub-dose prevention during the injection process.

### SUMMARY

### Technical Solutions

An objective of the present invention is to provide a quantitative injection pen and a method for its use. A reset rod is rotated in a reset direction to a pre-injection angle to switch to an injection state, and is subsequently rotated in an injection direction to quantitatively administer a medicament. A quantitative system is provided within the injection pen to ensure a specific dosage is delivered per injection. Notably, when the reset rod has not been fully rotated to the pre-injection angle, it can be rotated in reverse, returning along the injection direction to a stored state, without administering the medicament during this process, thereby providing an option to abort the current injection procedure.“

The present invention addresses the above problem by adopting the following technical solution: A quantitative injection pen, for delivering a predetermined dose of a medicament, comprising:
a lower housing, configured to accommodate a cartridge containing a medicament, wherein a distal end of the lower housing is configured for mounting an injection needle;
a push rod, having a distal end for urging a piston within the cartridge to inject the medicament, wherein the push rod is coaxially provided with a drive thread, and a rotation groove penetrating through each turn of the drive thread is provided along the axial direction of the push rod;
a cycle wheel, wherein an inner side of a distal portion of the cycle wheel is provided with a rotation rib engaged with the rotation groove;
an upper housing, provided with a threaded hole for engaging with the drive thread, and a fixed ratchet located inside the upper housing; wherein a distal side of the cycle wheel is provided with distal ratchet teeth engaged with the fixed ratchet and configured to drive the cycle wheel to rotate in an injection direction;
a reset rod, wherein a distal side of the reset rod is provided with ratchet slots; wherein a proximal side of the cycle wheel is provided with proximal ratchet teeth engaged with the ratchet slots, the proximal ratchet teeth being configured to drive the ratchet slots to rotate in the injection direction.

In a further preferred embodiment, the quantitative injection pen further comprises an activation sleeve for rotating the reset rod. An inner circumferential surface of the activation sleeve is provided with a guide thread, and an outer circumferential surface of the reset rod is provided with a guide thread groove engaged with the guide thread.

In a further preferred embodiment, an outer circumferential surface of the activation sleeve is axially provided with a guide protrusion. An inner circumferential surface of the upper housing is axially provided with a guide groove for engaging with the guide protrusion. In a further preferred embodiment,

[symbol]

In a further preferred embodiment, a distal side of the upper housing is provided with a cycle wheel buckle. A distal side of the cycle wheel is provided with a housing annular rib for engaging with the cycle wheel buckle.

In a further preferred embodiment, the cycle wheel buckle has a steep surface at its distal end and a gradual surface at its proximal end.

In a further preferred embodiment, the distal side of the upper housing is further provided with an upper housing cantilever for mounting the cycle wheel buckle, said cantilever having radial elasticity.

In a further preferred embodiment, an outer circumferential surface on the distal side of the reset rod is provided with a reset rod circumferential rib. The proximal side of the cycle wheel is provided with a reset rod buckle for engaging with the reset rod circumferential rib.

In a further preferred embodiment, the reset rod buckle has a steep surface at its distal end and a gradual surface at its proximal end.

In a further preferred embodiment, the cycle wheel, located on the distal side of the reset rod buckle, is provided with a circumferential rib retraction port for accommodating the reset rod circumferential rib.

In a further preferred embodiment, the distal side of the upper housing is further provided with a lower housing buckle. The lower housing is provided with an upper engagement hole for engaging with the lower housing buckle.

In a further preferred embodiment, the lower housing buckle has a gradual surface at its distal end and a steep surface at its proximal end.

In a further preferred embodiment, the distal side of the reset rod is provided with a semi-circumferential rib. The upper housing is internally provided with a circumferential limiting block for circumferentially engaging the semi-circumferential rib.

In a further preferred embodiment, the proximal side of the upper housing is provided with an indicator window. The inner circumferential surface of the activation sleeve is provided with an identification mark located on the same side as the indicator window.

In a further preferred embodiment, the proximal side of the push rod is provided with a conical step that tapers toward the distal end. The reset rod is internally provided with a conical surface that tapers toward the distal end and is configured to engage the conical step.

In a further preferred embodiment, the lower housing is internally provided with a cartridge retainer for securing the cartridge. The cartridge retainer comprises an annular body, a pressing buckle disposed on the annular body for securing the cartridge, and an arched rib disposed on the annular body for abutting against the distal end face of the upper housing.

In a further preferred embodiment, the cartridge retainer is provided with a hole-position buckle for engaging with the upper engagement hole.

In a further preferred embodiment, the hole-position buckle has a gradual surface at its distal end and a steep surface at its proximal end.

In a further preferred embodiment, the lower housing is further provided with a retraction groove to allow the pressing buckle to move radially outward.

In a further preferred embodiment, the lower housing is further provided with a lower engagement hole located on the distal side of the upper engagement hole and configured to engage with the hole-position buckle. When the hole-position buckle is engaged with the upper engagement hole, the pressing buckle is located at the same height as the retraction groove. When the hole-position buckle is engaged with the lower engagement hole, the pressing buckle is disengaged from the retraction groove.

In a further preferred embodiment, the distal end of the push rod is provided with an engagement step, and a fixed wheel part mounted on the engagement step for lifting the piston.

In a further preferred embodiment, the fixed wheel part comprises a drum-shaped block, a step socket disposed on the drum-shaped block for mounting the engagement step, a rod socket for engaging with the distal end of the push rod, and a rotation hole provided at the end of the rod socket and coaxial with the push rod.

In a further preferred embodiment, the rod socket is radially inwardly contracted.

In a further preferred embodiment, the distal side of the drum-shaped block is provided with a plurality of friction protrusions for abutting against the proximal end side of the piston.

In a further preferred embodiment, the lower housing is further provided with an observation window located on one side of the cartridge.

In a further preferred embodiment, the quantitative injection pen further comprises a pen cap; and the outer circumferential surface of the lower housing is further provided with a cap buckle for engaging with the pen cap.

In a further preferred embodiment, the distal side of the lower housing is further provided with an interface for mounting an injection needle.

In a further preferred embodiment, the proximal side of the activation sleeve is further provided with an activation button.

In a further preferred embodiment, the fixed ratchet is arranged around the threaded hole.

In a further preferred embodiment, the fixed ratchet comprises a large ratchet tooth and a small ratchet tooth.

A method for using the quantitative injection pen comprises the following steps:
S01. After the injection needle is inserted into the injection site, rotate the reset rod in a reset rotation direction until it can no longer rotate.
S02. Rotate the reset rod in an injection rotation direction until it can no longer rotate.

In a further preferred embodiment, a method for using a quantitative injection pen having an activation sleeve comprises the following steps:
S11. After the injection needle is inserted into the injection site, lift the activation sleeve in the proximal direction until it can no longer be lifted.
S12. Press the activation sleeve in the distal direction until it can no longer be pressed.

### Positive Effects

In summary, the present invention also offers the following advantages:
1. During the process of pulling the activation sleeve, for example, when it is pulled to 70% of the injection position, the user can push the activation sleeve back into the upper housing to abort the injection, without being forced to complete it. This provides an opportunity to cancel the procedure mid-injection, thereby enhancing the user-friendliness and high fault tolerance of the quantitative injection pen.
2. The push-pull operation of the activation sleeve can be performed single-handedly, further facilitating the injection process.
3. The design of the cartridge retainer ensures the cartridge is securely installed in the lower housing, guaranteeing a consistent dosage per injection.
4. The distal end of the push rod is equipped with a fixed wheel part, which functions similarly to a bearing, resulting in smoother operation when pushing the cartridge piston and preventing jamming caused by direct contact and friction between the push rod and the piston.
5. The large and small tooth design of the fixed ratchet provides an audible signal to remind the patient that the injection is complete.
6. The proximal end of the push rod is provided with a conical step, which prevents further distal movement when the medication in the cartridge is nearly depleted. This ensures a residual amount of medication remains in the cartridge, preventing a sub-dose injection due to insufficient medication during the final injection.

### DESCRIPTION OF THE DRAWINGS

FIG. 1 is an exploded schematic view of the quantitative injection pen according to the present invention.

FIG. 2a is a schematic structural view of the quantitative injection pen.

FIG. 2b is a schematic view of the injection pen in FIG. 2a with the pen cap removed.

FIG. 2c is a schematic view of the assembly in FIG. 2b with the lower housing removed.

FIG. 2d is a schematic view of the assembly in FIG. 2c with the upper housing removed.

FIG. 2e is a schematic view of the assembly in FIG. 2d with the activation sleeve removed.

FIG. 2f is a schematic view of the assembly in FIG. 2e with the cartridge removed.

FIG. 2g is a schematic view of the assembly in FIG. 2f with the cartridge retainer and the cycle wheel removed.

FIG. 3a is a schematic structural view of the lower housing.

FIG. 3b is a schematic view of the lower housing from a left-inclined perspective.

FIG. 3c is a schematic view of the lower housing from a right-inclined perspective.

FIG. 4a is a schematic structural view of the push rod.

FIG. 4b is a schematic structural view of the fixed wheel part.

FIG. 4c is a schematic view of the fixed wheel part from an upward-inclined perspective.

FIG. 5a is a schematic structural view of the cycle wheel.

FIG. 5b is a schematic view of the axial cross-section of the cycle wheel in FIG. 5a.

FIG. 5c is a schematic view of the cycle wheel from a left-inclined perspective.

FIG. 5d is a schematic view of the cycle wheel from a right-inclined perspective.

FIG. 5e is a schematic view of the cycle wheel observed from the distal end towards the proximal end.

FIG. 5f is a schematic view of the cycle wheel observed from the proximal end towards the distal end.

FIG. 6a is a schematic structural view of the upper housing.

FIG. 6b is a schematic view of the upper housing from a right-inclined perspective.

FIG. 6c is a schematic view of the upper housing observed from the proximal end towards the distal end.

FIG. 7a is a schematic structural view of the reset rod.

FIG. 7b is a schematic view of the axial cross-section of the reset rod in FIG. 7a.

FIG. 7c is a schematic view of the reset rod from a right-inclined perspective.

FIG. 7d is a schematic view of the reset rod observed from the distal end towards the proximal end.

FIG. 7e is a schematic view of the reset rod observed from the proximal end towards the distal end.

FIG. 8a is a schematic structural view of the activation sleeve.

FIG. 8b is a schematic view of the activation sleeve from an inclined perspective.

FIG. 8c is a schematic view of the activation sleeve observed from the distal end towards the proximal end.

FIG. 9 is a schematic structural view of the cartridge retainer.

FIG. 10a is a schematic view of a radial cross-section of the quantitative injection pen taken at the contact point between the guide thread groove and the guide thread.

FIG. 10b is a schematic view of the cross-sectional area in FIG. 10a.

Fig. 11a is a schematic view of a radial cross-section of the quantitative injection pen taken at the contact point between the ratchet slot and the proximal ratchet tooth.

FIG. 11b is a schematic view of the cross-sectional area in FIG. 11a.

FIG. 12a is a schematic view of a radial cross-section of the quantitative injection pen taken at the contact point between the fixed ratchet and the distal ratchet tooth.

FIG. 12b is a schematic view of the cross-sectional area in FIG. 12a.

In the drawings, the components represented by the reference numerals are as follows:

Devices used in conjunction with the quantitative pen include: a cartridge A, and a piston A1.

Lower housing 1, push rod 2, cycle wheel 3, upper housing 4, reset rod 5, activation sleeve 6, cartridge retainer 7, pen cap 8.

The lower housing 1 is provided with: an upper engagement hole 101, a retraction groove 102, a lower engagement hole 103, an observation window 104, a cap buckle 105, and an interface 106.

The push rod 2 is provided with: a drive thread 201, a rotation groove 202, an engagement step 203, a fixed wheel part 204, and a tapered step 205.

The cycle wheel 3 is provided with: a rotation rib 301, a distal ratchet tooth 302, a proximal ratchet tooth 303, a housing annular rib 304, a reset rod buckle 305, and a circumferential rib retraction port 306.

The upper housing 4 is provided with: a threaded hole 401, a fixed ratchet 402, a guide groove 403, a cycle wheel buckle 404, an upper housing cantilever 405, a lower housing buckle 406, a circumferential limiting block 407, and an indicator window 408.

The reset rod 5 is provided with: a ratchet slot 501, a guide thread groove 502, a reset rod circumferential rib 503, a semi-circumferential rib 504, and a conical surface 505.

The activation sleeve 6 is provided with: a guide thread 601, a guide protrusion 602, an identification mark 603, and an activation button 604.

The cartridge retainer 7 comprises: an annular body 701, a pressing buckle 702, an arched rib 703, and a hole-position buckle 704.

The fixed wheel part 204 comprises: a drum-shaped block 204a, a step socket 204b, a rod socket 204c, a rotation hole 204d, and a friction protrusion 204e.

The fixed ratchet 402 comprises: a large ratchet tooth 402a, and a small ratchet tooth 402b.

### DETAILED DESCRIPTION OF THE EMBODIMENTS

### Definitions

“Distal end” and “Proximal end”: When a user operates the quantitative injection pen, the pen is positioned between the user and the injection site. The end of the pen or its components closer to the injection site is referred to as the distal end, while the end closer to the user is referred to as the proximal end. It should be noted that “proximal” and “distal” are relative concepts.

“Injection rotation direction”: When a component is rotated in the injection direction, it drives the piston within the cartridge to move distally, thereby initiating the injection process. This rotation direction places the injector in an injection state.

“Reset rotation direction”: When a component is rotated in the reset direction, no injection occurs, and this action serves a reset function. This rotation direction causes the injector to exit the injection state and return to a state ready for the next injection. The reset rotation direction is opposite to the injection rotation direction.

“Steep surface” and “Gradual surface”: In general mechanical design, for instance, in ratchet mechanisms often used to restrict unidirectional movement or achieve stepped movement of components, one side of the ratchet tooth has a steep profile to block movement in that direction; this side is termed the steep surface. The other side has a gentle, sloping profile to allow sliding movement in that direction; this side is termed the gradual surface. Similarly, buckles functioning akin to ratchet structures also possess the aforementioned steep surface and gradual surface.

The present invention is described in further detail below with reference to the accompanying drawings.

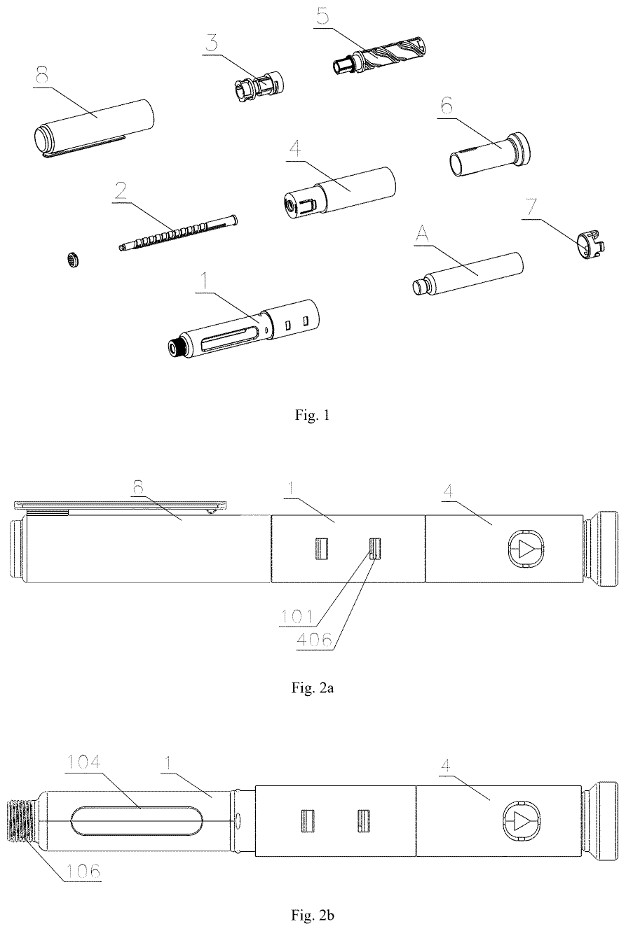

Referring to FIG. 1, an exploded schematic view of the quantitative injection pen is shown, comprising a lower housing 1, a push rod 2, a cycle wheel 3, an upper housing 4, a reset rod 5, an activation sleeve 6, a cartridge retainer 7, and a pen cap 8.

The production process of the quantitative injection pen is carried out on an assembly line. The installation process specifically comprises the following steps:
S21. Assembling the cycle wheel 3 with the reset rod 5 to form a first sub-assembly;
S22. Assembling the first sub-assembly with the activation sleeve 6 to form a second sub-assembly;
S23. Assembling the second sub-assembly with the upper housing 4 to form a third sub-assembly;
S24. Assembling the third sub-assembly with the push rod 2 to form a fourth sub-assembly;
S25. Assembling the pen cap 8, the cartridge retainer 7, and the lower housing 1 to form a fifth sub-assembly;
S26. Assembling the fourth sub-assembly with the fifth sub-assembly to form the quantitative injection pen;
S27. Performing primary packaging and secondary packaging of the quantitative injection pen, affixing labels to each package, and storing the products after passing qualification inspection.

Generally, the assembled quantitative injection pen described above is a single-use product, intended to be disposed of after use, although non-disposable applications are not precluded.

The present invention is described in further detail below with reference to the accompanying drawings.

Referring to FIG. 1, an exploded schematic view of the quantitative injection pen is shown, comprising a lower housing 1, a push rod 2, a cycle wheel 3, an upper housing 4, a reset rod 5, an activation sleeve 6, a cartridge retainer 7, and a pen cap 8.

The production process of the quantitative injection pen is carried out on an assembly line. The installation process specifically comprises the following steps:
S21. Assembling the cycle wheel 3 with the reset rod 5 to form a first sub-assembly;
S22. Assembling the first sub-assembly with the activation sleeve 6 to form a second sub-assembly;
S23. Assembling the second sub-assembly with the upper housing 4 to form a third sub-assembly;
S24. Assembling the third sub-assembly with the push rod 2 to form a fourth sub-assembly;
S25. Assembling the pen cap 8, the cartridge retainer 7, and the lower housing 1 to form a fifth sub-assembly;
S26. Assembling the fourth sub-assembly with the fifth sub-assembly to form the quantitative injection pen;
S27. Performing primary packaging and secondary packaging of the quantitative injection pen, affixing labels to each package, and storing the products after passing qualification inspection.

Generally, the assembled quantitative injection pen described above is a single-use product, intended to be disposed of after use, although non-disposable applications are not precluded.

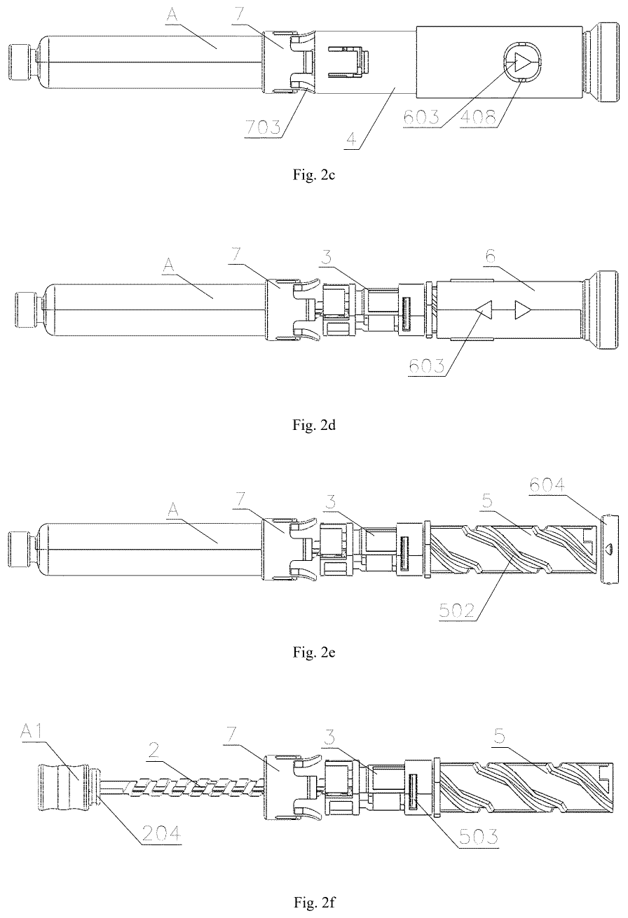

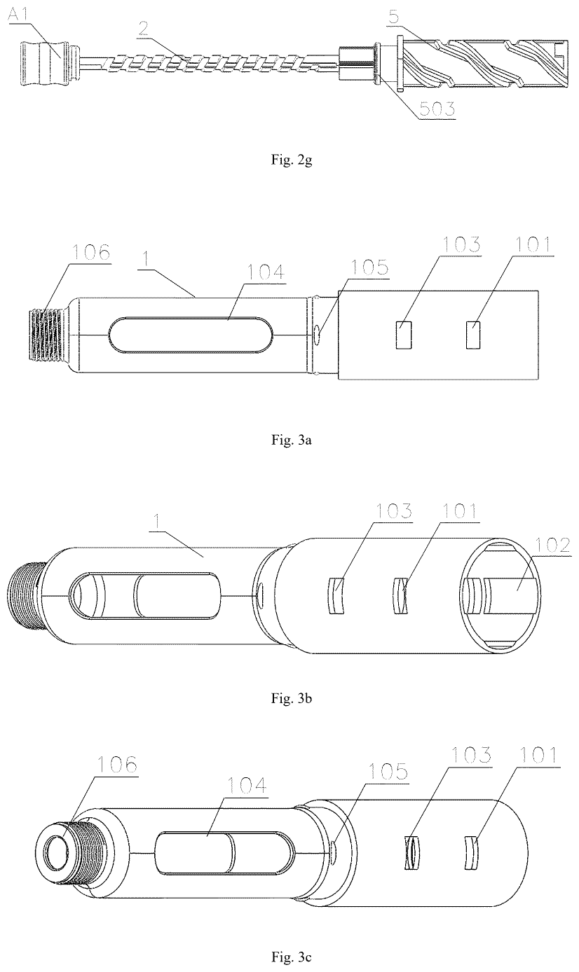

Referring to FIG. 2a, the assembled quantitative injection pen is shown. From left to right are the pen cap 8, the lower housing 1, and the upper housing 4. The pen cap 8 is sleeved over the distal portion of the lower housing 1, and the lower housing 1 is sleeved over the distal portion of the upper housing 4. It can also be seen in the figure that at the location where the lower housing 1 sleeves the upper housing 4, the lower housing buckle 406 engages the upper engagement hole 101, thereby achieving axial and circumferential fixation between the lower housing 1 and the upper housing 4.

Referring to FIG. 2b, a schematic view of the quantitative injection pen with the pen cap 8 removed, exposing the distal portion of the lower housing 1, is shown. The distal portion of the lower housing 1 is provided with an observation window 104 and an interface 106. The observation window 104 allows for viewing how much liquid medicament remains in the cartridge A inside the lower housing 1, enabling timely judgment on whether continued use is possible or if disposal is required. The exterior of the interface 106 is provided with threads compatible with an injection needle, used for replacing the injection needle between two injection procedures. The specific steps are as follows:
S31. Ensure the injection pen is reset to prevent medicament leakage during the needle replacement process.
S32. Locate the needle interface 106 at the tail end of the injection pen housing. Select a needle specification compatible with this quantitative injection pen, ensuring the chosen needle is compatible with the connection port of the pen's interface.
S33. Remove the original needle, typically by unscrewing it counterclockwise.
S34. Align the new needle with the injection pen interface 106, rotate to secure it firmly, and finally ensure the connection is completely tight.

Referring to FIG. 2c, a schematic view of the quantitative injection pen with the activation sleeve 6 removed, exposing the sleeved reset rod 5, is shown. It can be seen that after the lower housing 1 and the upper housing 4 are axially fixed, the distal end face of the upper housing 4 presses against the arched rib 703 on the cartridge retainer 7. This imparts an elastic force on the cartridge retainer 7 tending to move it distally. This elastic force acts on the proximal end face of the cartridge A, enabling the cartridge A to be more securely fixed within the lower housing 1, meaning it will not move during injection, thereby ensuring the injection dosage remains stable at the specified value.

Referring to FIG. 2d, a schematic view of the quantitative injection pen with the upper housing 4 removed, exposing the sleeved cycle wheel 3 and the activation sleeve 6, is shown. The side surface of the activation sleeve 6 is provided with an identification mark 603, which comprises two parts: a downward-pointing arrow indicating the distal direction and an upward-pointing arrow indicating the proximal direction. When the activation sleeve 6 is in the non-injection state, the upward arrow appears in the indicator window 408, as shown in FIG. 2c, indicating that the activation sleeve 6 should be pulled proximally to prepare for injection. It can be understood that when the activation sleeve 6 is pulled to its utmost proximal position, the downward arrow appears in the indicator window 408, indicating that the activation sleeve 6 should be pressed distally to perform the injection.

Referring to FIG. 2e, a schematic view of the quantitative injection pen with the activation sleeve 6 removed, exposing the reset rod 5, is shown. The activation button 604 located above the activation sleeve 6 is retained in the view. The activation button 604 is mounted embedded in the proximal side of the activation sleeve 6 to provide a pressing aid for the user. The outer circumferential surface of the reset rod 5 is provided with a guide thread groove 502 extending axially and helically, which engages with the guide thread 601 inside the activation sleeve 6.

Referring to FIG. 2f, a schematic view of the quantitative injection pen with the cartridge A removed, exposing the distal portion of the push rod 2, is shown. It can be seen that a fixed wheel part 204 is provided at the distal end of the push rod 2, used for urging the piston Al inside the cartridge A. When the piston Al moves distally, it expels a volume of medicament equivalent to its displacement volume for injection.

Referring to FIG. 2g, a schematic view of the quantitative injection pen after removal of the cartridge retainer 7 and the cycle wheel 3, exposing the proximal portion of the push rod 2, is shown. It can be seen that the reset rod 5 is axially engaged and fixed to the cycle wheel 3 via the reset rod circumferential rib 503.

Referring to FIG. 3a, the specific structure of the lower housing 1 is shown. The distal end of the lower housing 1 is provided with an interface 106 for mounting an injection needle. Proximal to the interface 106 is an observation window 104, having a length similar to that of the cartridge A. Typically, the observation window 104 is provided on both the left and right sides of the lower housing 1. Distal to the observation window 104 is a cap buckle 105 for engaging the pen cap 8. Proximal to the cap buckle 105, a lower engagement hole 103 and an upper engagement hole 101 are sequentially arranged. Usually, the lower engagement hole 103 and the upper engagement hole 101 are also provided on both the left and right sides of the lower housing 1.

Referring to FIG. 3b, a schematic view of the lower housing 1 tilted at a certain angle is shown. It can be observed that the inner side of the lower housing 1 is provided with a retraction groove 102. Typically, the retraction groove 102 is provided on both the left and right sides of the lower housing 1.

Referring to FIG. 3c, a schematic view of the lower housing 1 tilted in another direction is shown. It can be observed that the interface has pores communicating with the interior. The gaps between these pores can be filled with a sealing material to prevent medicament leakage after the injection needle is mounted onto the cartridge A.

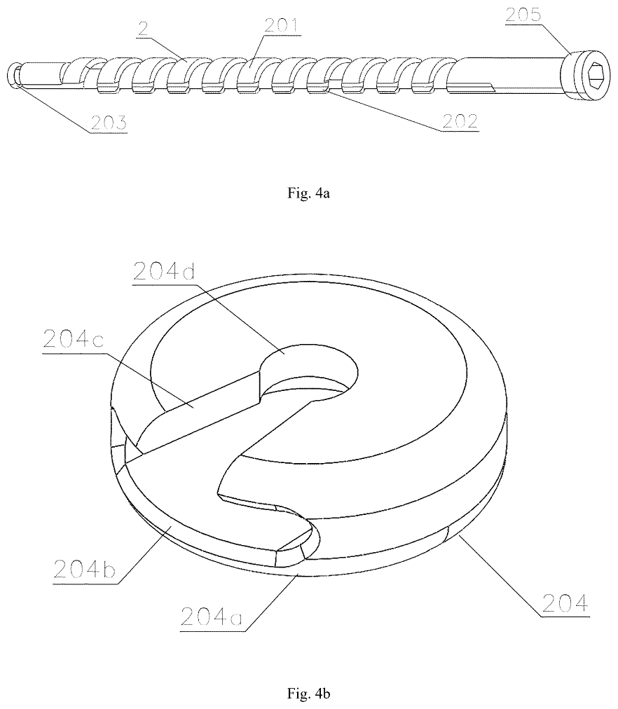

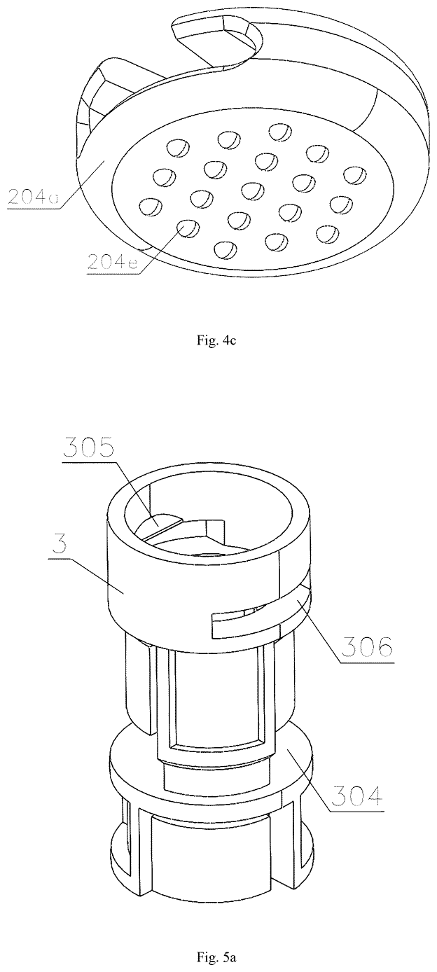

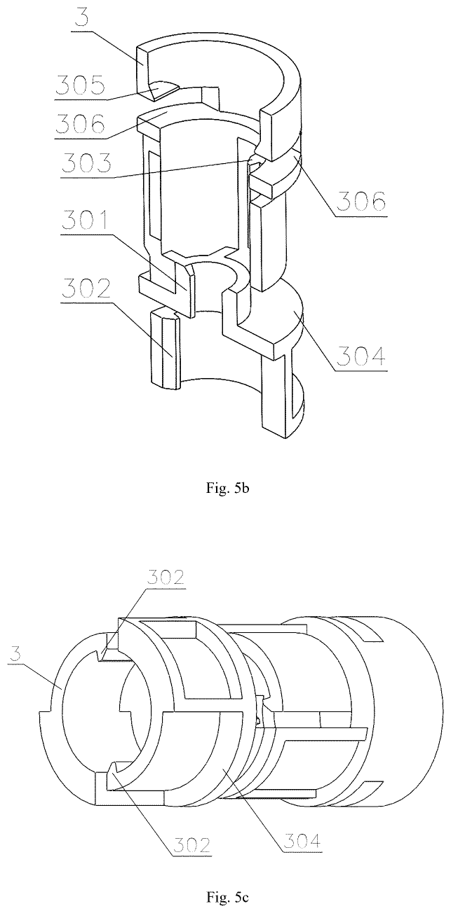

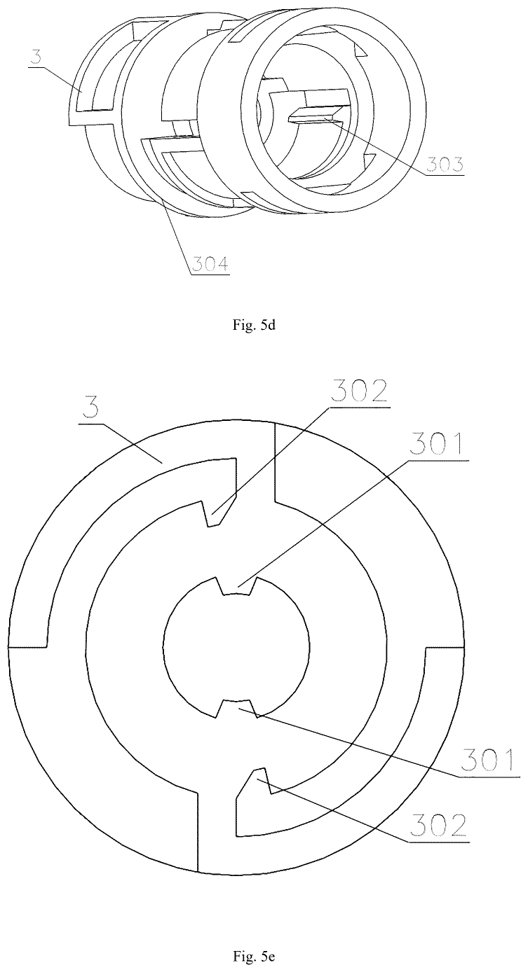

Referring to FIG. 4a, a schematic structural view of the push rod 2 is shown. It can be seen that an engagement step 203 for mounting the fixed wheel part 204 is provided at the very distal end of the push rod 2. A drive thread 201 extends axially and helically along the outer circumferential surface of the push rod 2. A rotation groove 202 also extends axially and penetrates through each turn of the drive thread 201. A conical step 205, which tapers towards the distal end, is provided at the very proximal end of the push rod 2.

Referring to FIG. 4b, the specific structure of the fixed wheel part 204 is shown. The fixed wheel part 204 is substantially a drum-shaped block 204a with a central cavity. A rotation hole 204d for mounting the distal end of the push rod 2 is provided in the center of the upper end of the drum-shaped block 204a. A rod socket 204c, which extends radially and communicates with the rotation hole 204d, is also provided at the upper end of the drum-shaped block 204a. A step socket 204b for mounting the engagement step 203 and communicating with the rod socket 204c is provided on the lateral side of the drum-shaped block 204a.

Referring to FIG. 4c, a schematic view of the fixed wheel part 204 tilted upward is shown. It can be seen that the lower end of the drum-shaped block 204a is provided with a plurality of uniformly distributed friction protrusions 204e. This ensures that after the lower end of the drum-shaped block 204a contacts the piston Al inside the cartridge A, relative stillness is achieved through friction. Furthermore, the distal end of the push rod 2 can rotate freely within the rotation hole 204d.

The specific method for mounting the fixed wheel part 204 onto the distal end of the push rod 2 is as follows: align the engagement step 203 with the step socket 204b and push radially inward, causing the distal end of the push rod 2 to slide through the rod socket 204c into the rotation hole 204d, where it can then rotate freely. The rod socket 204c is radially inwardly contracted, thereby securing the distal end of the push rod 2 within the rotation hole 204d and preventing it from easily sliding out through the rod socket 204c.

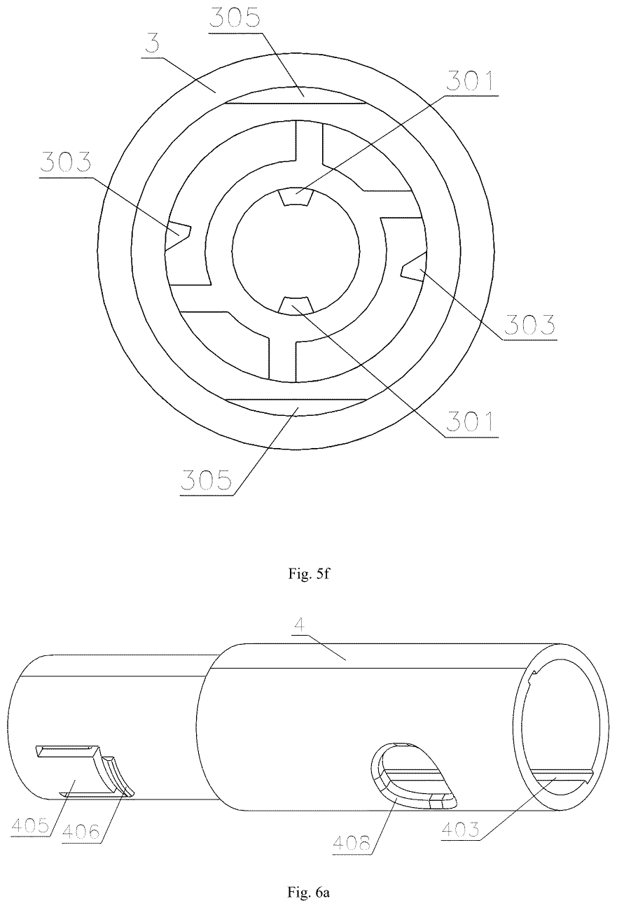

Referring to FIG. 5a, the specific structure of the cycle wheel 3 is shown. Visible in the figure, from bottom to top, are a housing annular rib 304, and a circumferential rib retraction port 306 provided on the cycle wheel 3, typically provided on both the left and right sides of the cycle wheel 3. Above the circumferential rib retraction port 306 on the inner side of the cycle wheel 3, a reset rod buckle 305 is also provided.

Referring to FIG. 5b, a schematic view of the vertical cross-section of the cycle wheel 3 is shown to illustrate its internal structure. Visible in the figure, from bottom to top, are distal ratchet teeth 302, typically provided on both the left and right sides of the cycle wheel 3; a rotation rib 301 located above the distal ratchet teeth 302, situated on the inner surface of a reserved hole in the cycle wheel 3 for accommodating the push rod 2, and used for engaging with the rotation groove 202 on the push rod 2. That is, when the rotation rib 301 engages with the rotation groove 202, the cycle wheel 3 and the push rod 2 achieve circumferential limiting, meaning the cycle wheel 3 can drive the push rod 2 to rotate in either direction.

Proximal ratchet teeth 303 are provided above the rotation rib 301, typically provided on both the left and right sides of the cycle wheel 3, with the gradual surfaces and steep surfaces of the two proximal ratchet teeth 303 facing the same direction. The circumferential rib retraction port 306 is provided above the proximal ratchet teeth 303, and the reset rod buckle 305 is provided above the circumferential rib retraction port 306.

Referring to FIG. 5c, a schematic view of the cycle wheel 3 from an inclined perspective is shown. It can be seen that the distal ratchet teeth 302 are provided one on each of the left and right sides of the cycle wheel 3, with the gradual surfaces and steep surfaces of the two distal ratchet teeth 302 facing the same direction. Connected to the two distal ratchet teeth 302 is the housing annular rib 304.

Referring to FIG. 5d, a schematic view of the cycle wheel 3 tilted in another direction is shown. It can be seen that the proximal ratchet teeth 303 are provided one on each of the left and right sides of the cycle wheel 3, with the gradual surfaces and steep surfaces of the two proximal ratchet teeth 303 facing the same direction.

Referring to FIG. 5e, a schematic view of the cycle wheel 3 observed from the distal end towards the proximal end is shown. It can be seen that the two distal ratchet teeth 302 are arranged 180 degrees apart, and the two rotation ribs 301 are also arranged 180 degrees apart, for engaging with the two rotation grooves 202, respectively.

Referring to FIG. 5f, a schematic view of the cycle wheel 3 observed from the proximal end towards the distal end is shown. It can be seen that the two proximal ratchet teeth 303 are arranged 180 degrees apart, and the two reset rod buckles 305 are also arranged 180 degrees apart.

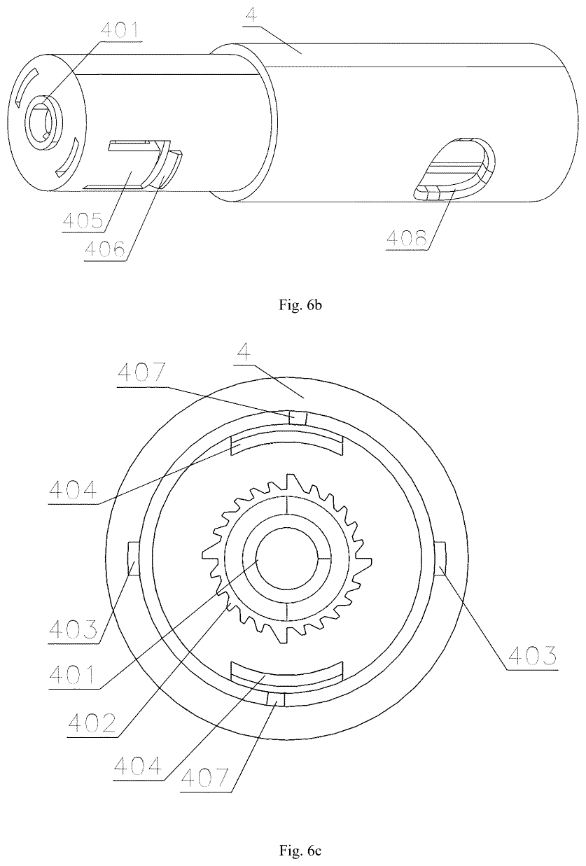

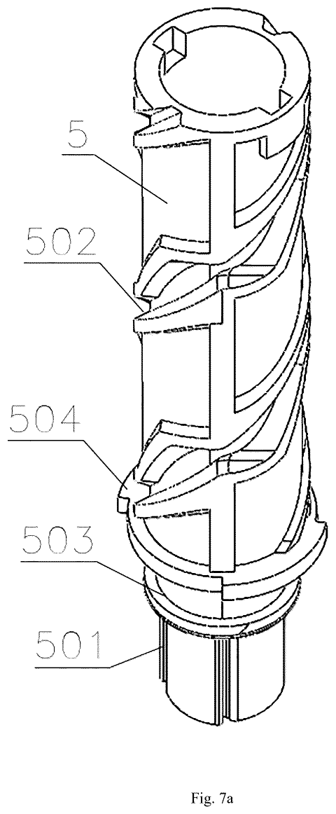

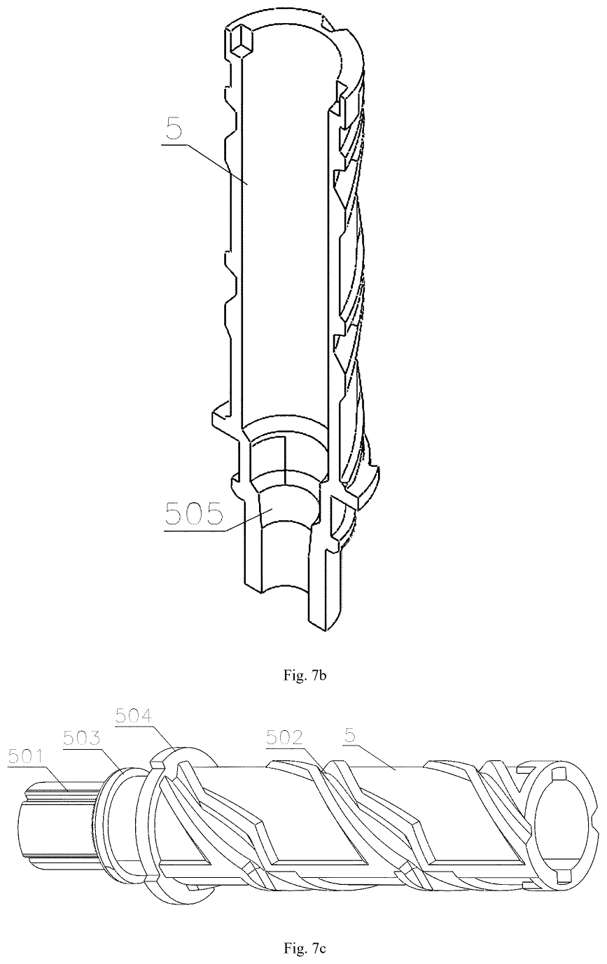

Referring to FIG. 6a, a schematic structural view of the upper housing 4 is shown. The upper housing 4 is provided with an upper housing cantilever 405, typically one on each of the left and right sides. A lower housing buckle 406 is provided at the proximal end of each upper housing cantilever 405. The lower housing buckle 406 has a gradual surface at its distal end and a steep surface at its proximal end, for engaging with the upper engagement hole 101 on the lower housing 1 at the distal side, thereby achieving both axial and circumferential fixation between the lower housing 1 and the upper housing 4. An indicator window 408 is provided proximal to the lower housing buckle 406. A guide groove 403 is provided on the inner side of the upper housing 4, typically one on each of the left and right inner sides.

Referring to FIG. 6b, a schematic structural view from another perspective is shown. It can be seen that a threaded hole 401 is provided on the distal side of the upper housing 4 for threadedly engaging with the drive thread 201.

Referring to FIG. 6c, a schematic structural view of the upper housing 4 observed from the proximal end towards the distal end is shown. It can be seen that a fixed ratchet 402, concentric with the threaded hole 401, is arranged around the threaded hole 401. The upper housing 4 is also provided with a cycle wheel buckle 404 for engaging with the housing annular rib 304 of the cycle wheel 3, thereby axially fixing the cycle wheel 3 to the upper housing 4. The cycle wheel buckles 404 are provided one each on the upper and lower sides of the inner surface of the upper housing 4, arranged 180 degrees apart. A guide groove 403 is also provided inside the upper housing 4. The guide grooves 403 are provided one each on the left and right sides of the inner surface of the upper housing 4, arranged 180 degrees apart.

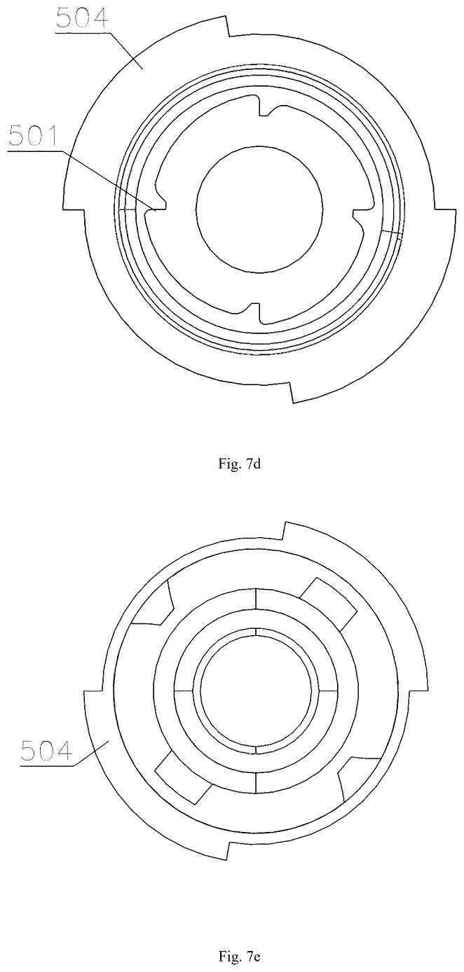

Referring to FIG. 7a, a schematic structural view of the reset rod 5 is shown. The distal end of the reset rod 5 is provided with ratchet slots 501. Four ratchet slots 501 are provided on the reset rod 5, arranged at 90-degree intervals. Proximal to the ratchet slots 501 is provided a reset rod circumferential rib 503. The reset rod circumferential rib 503 is for engaging with the reset rod buckle 305 of the cycle wheel 3, thereby axially fixing the reset rod 5 to the cycle wheel 3. A guide thread groove 502 extending axially and helically is provided proximal to the reset rod circumferential rib 503.

Referring to FIG. 7b, a schematic view of the axial cross-section of the reset rod 5 is shown. The distal side of the reset rod 5 is provided with a conical surface 505 that tapers towards the distal end. Its function is that during the distal movement of the push rod 2, when nearly all the medicament is about to be injected, the conical step 205 will catch on the conical surface 505, thereby preventing further distal movement of the push rod 2. The advantage of this is that it ensures a residual amount of medicament remains in the cartridge A, preventing a sub-dose injection caused by insufficient medicament during the final injection procedure.

Referring to FIG. 7c, a schematic structural view of the reset rod 5 from another perspective is shown.

Referring to FIG. 7d, a schematic structural view of the reset rod 5 observed from the distal end towards the proximal end is shown. It can be seen that four ratchet slots 501 are provided at the distal end of the reset rod 5, arranged at 90-degree intervals.

Referring to FIG. 7e, a schematic structural view of the reset rod 5 observed from the proximal end towards the distal end is shown. Semi-circumferential ribs 504 are provided one on each of the left and right sides of the reset rod 5, arranged 180 degrees apart. Each semi-circumferential rib 504 is fan-shaped and subtends an angle of approximately 90 degrees. That is, a sliding slot of approximately 90 degrees is defined between the two semi-circumferential ribs 504. The circumferential limiting block 407 can slide within this sliding slot, thereby restricting the rotation angle of the reset rod 5. By adjusting the subtended angle of this sliding slot, the maximum rotation angle of the reset rod 5 can be controlled, which in turn controls the travel distance of the push rod 2 per injection, thereby achieving dose control. This angle is set during manufacturing.

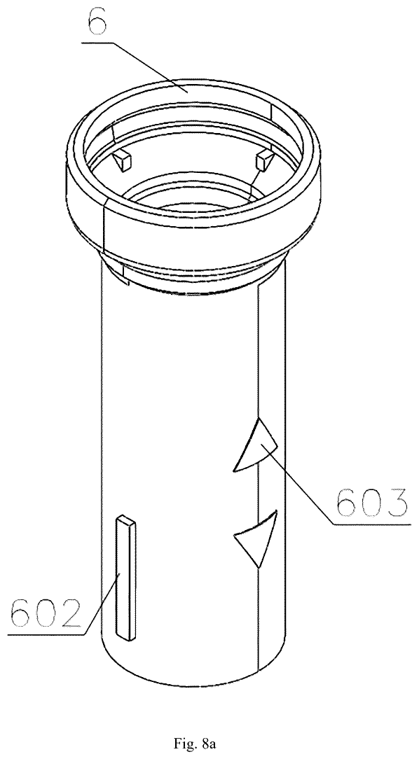

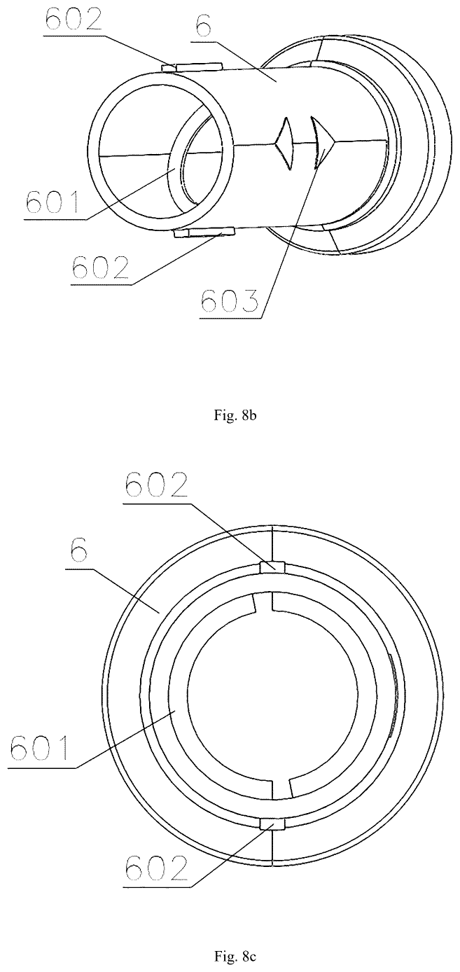

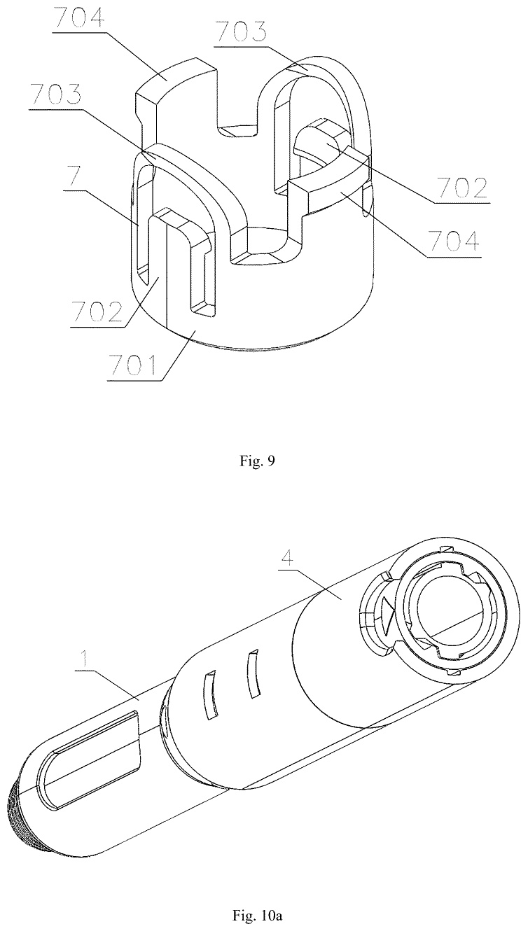

Referring to FIG. 8a, a schematic structural view of the activation sleeve 6 is shown. The outer circumferential surface of the activation sleeve 6 is provided with guide protrusions 602. The guide protrusions 602 are provided one on each of the left and right sides of the outer circumferential surface. The guide protrusions 602 cooperate with the guide grooves 403 on the inner circumferential surface of the upper housing 4 to achieve circumferential fixation within the upper housing 4.

Referring to FIG. 8b, a schematic structural view of the activation sleeve 6 from another perspective is shown. The activation sleeve 6 is internally provided with a guide thread 601 that matches the guide thread groove 502. The activation sleeve 6 is threadedly engaged with the reset rod 5 via the guide thread 601. Since the rotation angle of the reset rod 5 is limited by the semi-circumferential ribs 504, the travel distance of the activation sleeve 6 between the distal and proximal ends is consequently also limited by the semi-circumferential ribs 504. This means that after assembly, the activation sleeve 6 cannot detach from the upper housing 4. Specifically, the travel distance of the activation sleeve 6 between the distal and proximal ends is equal to the length of the line connecting the midpoints of the two arrows of the identification mark 603.

Referring to FIG. 8c, a schematic structural view of the activation sleeve 6 observed from the distal end towards the proximal end is shown.

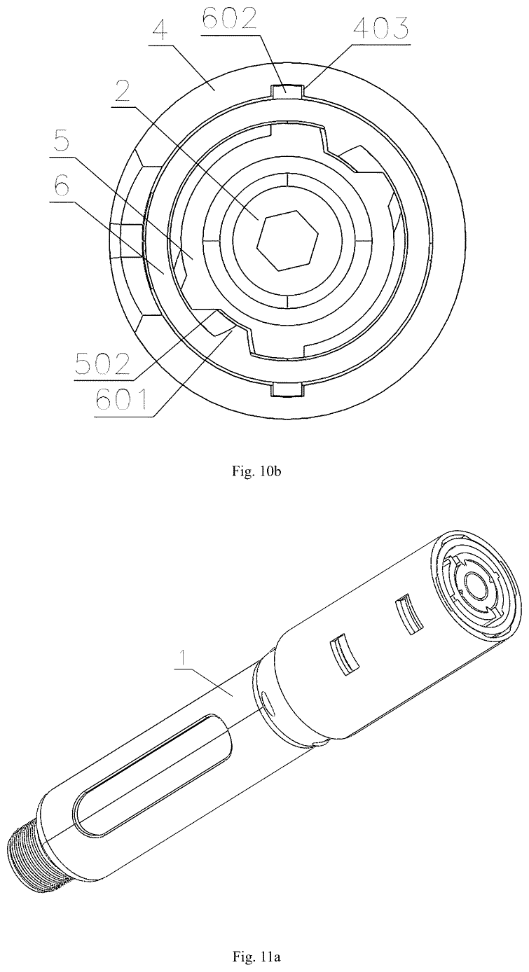

Referring to FIG. 9, a schematic view of the cartridge retainer 7 is shown. The method for installing the cartridge A and the cartridge retainer 7 into the lower housing 1 specifically comprises the following steps:
S41. Insert the annular body 701 of the cartridge retainer 7 into the lower housing 1 with one end facing distally. Adjust the angle of the cartridge retainer 7 so that the hole-position buckle 704 is installed into the upper engagement hole 101.
S42. Insert the cartridge A into the lower housing 1. Under the pressure from the cartridge A, the hole-position buckle 704 expands radially outward, with a portion of it entering the retraction groove 102.
S43. Continue to press the cartridge retainer 7 distally until the hole-position buckle 704 shifts from the upper engagement hole 101 to the lower engagement hole 103. At this point, the distal steep surface of the pressing buckle 702 snap-fits with the proximal end of the cartridge A.
S44. Insert the upper housing 4 into the lower housing 1 until the lower housing buckle 406 snap-fits into the upper engagement hole 101. At this point, the distal end face of the upper housing 4 presses against the arched rib 703, causing the distal steep surface of the pressing buckle 702 to fully contact the proximal end of the cartridge A.

Specifically, a preload exists between the arched rib 703 and the distal end face of the upper housing 4. Under this preload, the entire cartridge retainer 7 is subjected to a downward force. Consequently, the pressing buckle 702 on the cartridge retainer 7 presses downward on the cartridge A, ensuring the cartridge A remains stable during use.

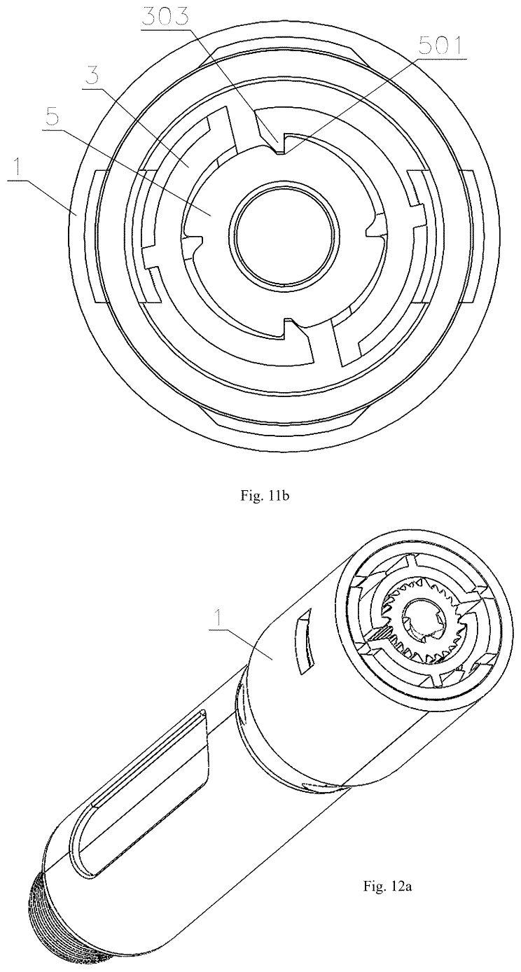

Referring to FIG. 10a, a schematic view of a radial cross-section of the quantitative injection pen taken at the contact point between the guide thread groove 502 and the guide thread 601 is shown.

Referring to FIG. 10b, a schematic view of the cross-sectional area in FIG. 10a is shown. The engagement between the guide thread groove 502 and the guide thread 601 is visible. When the activation sleeve 6 moves proximally, the reset rod 5 in the figure rotates clockwise, i.e., in the reset rotation direction. When the activation sleeve 6 moves distally, the reset rod 5 in the figure rotates counterclockwise, i.e., in the injection rotation direction. During the proximal and distal movement of the activation sleeve 6, guided by the cooperation between the guide groove 403 and the guide protrusion 602, it can only move axially. The angle of the guide thread groove 502 and the guide thread 601 is relatively gradual, allowing the pulling action on the activation sleeve 6 to drive the rotation of the reset rod 5.

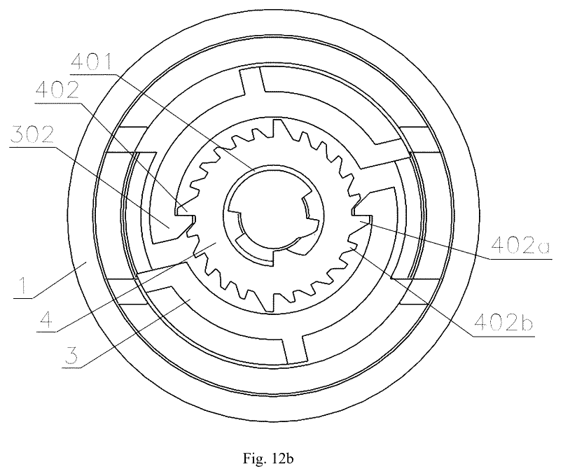

Referring to FIG. 11a, a schematic view of a radial cross-section of the quantitative injection pen taken at the contact point between the ratchet slot 501 and the proximal ratchet tooth 303 is shown.

Referring to FIG. 11b, a schematic view of the cross-sectional area in FIG. 11a is shown. It can be seen that when the reset rod 5 rotates counterclockwise (i.e., in the injection rotation direction), the ratchet slot 501 drives the proximal ratchet tooth 303 to rotate together counterclockwise (i.e., in the injection rotation direction). When the reset rod 5 rotates clockwise (i.e., in the reset rotation direction), the ratchet slot 501 does not drive the proximal ratchet tooth 303; instead, it overrides the currently engaged proximal ratchet tooth 303 and moves to the next proximal ratchet tooth 303. In summary, the reset rod 5 can drive the cycle wheel 3 to rotate counterclockwise (i.e., in the injection rotation direction), but cannot drive the cycle wheel 3 to rotate clockwise (i.e., in the reset rotation direction).

Notably, during the clockwise rotation of the ratchet slot 501 (e.g., when the proximal ratchet tooth 303 is located between two ratchet slots 501), the ratchet slot 501 can rotate counterclockwise back to its initial position corresponding to the original proximal ratchet tooth 303. This process does not drive the rotation of the proximal ratchet tooth 303. This mechanism allows the user, during the process of pulling the activation sleeve 6 proximally (for instance, when it is pulled to 70% of the injection position), to push the activation sleeve 6 directly back into the upper housing 4 to abort the injection, without being forced to complete it. This provides an opportunity to cancel the procedure mid-injection, thereby enhancing the user-friendliness and high fault tolerance of the quantitative injection pen.

Referring to FIG. 12a, a schematic view of a radial cross-section of the quantitative injection pen taken at the contact point between the fixed ratchet 402 and the distal ratchet tooth 302 is shown.

Referring to FIG. 12b, a schematic view of the cross-sectional area in FIG. 12a is shown. In the engaged state between the fixed ratchet 402 and the distal ratchet tooth 302, rotation of the distal ratchet tooth 302 is only permitted in the counterclockwise direction (i.e., the injection rotation direction), and is prevented in the clockwise direction (i.e., the reset rotation direction). In the clockwise direction, movement is blocked by the steep surfaces of the ratchet teeth. The purpose of this design is to prevent the reset rod 5 from driving the cycle wheel 3 to rotate clockwise when the reset rod 5 itself is rotating clockwise (i.e., in the reset rotation direction). Notably, the fixed ratchet 402 comprises four sets of ratchet teeth. Each set includes one large ratchet tooth 402a and five small ratchet teeth 402b. When the distal ratchet tooth 302 passes over the fixed ratchet 402, it produces different sounds as it passes the large ratchet tooth 402a and the five small ratchet teeth 402b. The sound produced by the large ratchet tooth 402a is louder. Thus, during the rotation of the cycle wheel 3 in the injection direction, a series of five consecutive softer “click” sounds followed by one louder “CLICK” sound is heard. When the louder “CLICK” sound is heard, it indicates that the injection is complete. This sound also serves as an audible indicator to remind the patient that the injection is finished.

In summary, the method for using the quantitative injection pen is as follows:
S01. After the injection needle is inserted into the injection site, rotate the reset rod 5 in the reset rotation direction until it can no longer rotate.
S02. Rotate the reset rod 5 in the injection rotation direction until it can no longer rotate.

When the activation sleeve 6 is installed, the method for using the quantitative injection pen is as follows:
S11. After the injection needle is inserted into the injection site, lift the activation sleeve 6 in the proximal direction until it can no longer be lifted.
S12. Press the activation sleeve 6 in the distal direction until it can no longer be pressed.

This specific embodiment is merely an illustration of the present invention and should not be construed as limiting the invention. Those skilled in the art, after reading this specification, can make modifications to this embodiment as needed, provided such modifications involve no inventive contribution. However, all such modifications falling within the scope of the claims of the present invention shall be protected under patent law.

### Embodiment 1

In the present embodiment, the structure of the quantitative injection pen comprises:

Devices used in conjunction with the quantitative pen: a cartridge A, and a piston A1.

Main components of the quantitative pen include: a lower housing 1, a push rod 2, a cycle wheel 3, an upper housing 4, a reset rod 5, a cartridge retainer 7, and a pen cap 8.

The lower housing 1 is provided with: two upper engagement holes 101, two retraction grooves 102, two lower engagement holes 103, two observation windows 104, four cap buckles 105, and an interface 106.

The push rod 2 is provided with: a drive thread 201, two rotation grooves 202, an engagement step 203, a fixed wheel part 204, and a conical step 205.

The cycle wheel 3 is provided with: two rotation ribs 301, two distal ratchet teeth 302, two proximal ratchet teeth 303, a housing annular rib 304, two reset rod buckles 305, and two circumferential rib retraction ports 306.

The upper housing 4 is provided with: a threaded hole 401, a fixed ratchet 402, two guide grooves 403, two cycle wheel buckles 404, two upper housing cantilevers 405, two lower housing buckles 406, two circumferential limiting blocks 407, and an indicator window 408.

The reset rod 5 is provided with: four ratchet slots 501, a guide thread groove 502, a reset rod circumferential rib 503, two semi-circumferential ribs 504, and a conical surface 505.

The fixed wheel part 204 comprises: a drum-shaped block 204a, a step socket 204b, a rod socket 204c, a rotation hole 204d, and nineteen friction protrusions 204e.

The fixed ratchet 402 comprises: four large ratchet teeth 402a and twenty small ratchet teeth 402b.

### Embodiment 2

In the present embodiment, the structure of the quantitative injection pen is substantially identical to that of Embodiment 1, with the difference lying in that the structure of the quantitative injection pen further comprises:
an activation sleeve 6.

The activation sleeve 6 is provided with: a guide thread 601, a guide protrusion 602, an identification mark 603, and an activation button 604.

In the present embodiment, the structure of the quantitative injection pen is substantially identical to that of Embodiment 1, with the difference lying in that: the lower housing 1 is further provided with: two retraction grooves 102.

the cartridge retainer 7 specifically comprises: an annular body 701, two pressing buckles 702, an arched rib 703, and a hole-position buckle 704.

Furthermore, wherever possible, the same or similar reference numerals are used in the drawings and the description to refer to the same or like parts or steps. The drawings are presented in a simplified form and are not necessarily drawn to scale. Directional terms such as top, bottom, left, right, up, above, over, below, beneath, behind, and front are used with reference to the figures for convenience and clarity only. These and similar directional terms should not be construed to limit the scope of the present disclosure in any manner.

1. A quantitative injection pen for delivering a predetermined dose of a medicament, characterized in that it comprises:
a lower housing configured to accommodate a cartridge containing a medicament, wherein a distal end of the lower housing is configured for mounting an injection needle;
a push rod, having a distal end for urging a piston within the cartridge (A) to inject the medicament, wherein the push rod is coaxially provided with a drive thread, and a rotation groove penetrating through each turn of the drive thread is provided along an axial direction of the push rod;
a cycle wheel, wherein an inner side of a distal portion of the cycle wheel is provided with a rotation rib engaged with the rotation groove;
an upper housing, provided with a threaded hole for engaging with the drive thread, and a fixed ratchet located inside the upper housing; wherein a distal side of the cycle wheel (3) is provided with a distal ratchet tooth engaged with the fixed ratchet and configured to drive the cycle wheel to rotate in an injection rotation direction;
a reset rod, wherein a distal side of the reset rod is provided with a ratchet slot; wherein a proximal side of the cycle wheel is provided with a proximal ratchet tooth engaged with the ratchet slot, the proximal ratchet tooth being configured to drive the ratchet slot to rotate in the injection rotation direction.

2. The quantitative injection pen according to claim 1, characterized in that, it further comprises an activation sleeve for rotating the reset rod, wherein an inner circumferential surface of the activation sleeve is provided with a guide thread; and an outer circumferential surface of the reset rod is provided with a guide thread groove engaged with the guide thread.

3. The quantitative injection pen according to claim 2, characterized in that, an outer circumferential surface of the activation sleeve is axially provided with a guide protrusion; and an inner circumferential surface of the upper housing is axially provided with a guide groove for engaging with the guide protrusion.

4. The quantitative injection pen according to claim 1, characterized in that, a distal side of the upper housing is provided with a cycle wheel buckle; and a distal side of the cycle wheel is provided with a housing annular rib for engaging with the cycle wheel buckle.

5. The quantitative injection pen according to claim 4, characterized in that, the cycle wheel buckle has a steep surface at its distal end and a gradual surface at its proximal end.

6. The quantitative injection pen according to claim 4, characterized in that, the distal side of the upper housing is further provided with an upper housing cantilever for mounting the cycle wheel buckle, said cantilever having radial elasticity.

7. The quantitative injection pen according to claim 1, characterized in that,
an outer circumferential surface on a distal side of the reset rod is provided with a reset rod circumferential rib; and a proximal side of the cycle wheel is provided with a reset rod buckle for engaging with the reset rod circumferential rib.

8. The quantitative injection pen according to claim 7, characterized in that, the reset rod buckle has a steep surface at its distal end and a gradual surface at its proximal end.

9. The quantitative injection pen according to claim 7, characterized in that, the cycle wheel is provided, on a distal side of the reset rod buckle, with a circumferential rib retraction port for accommodating the reset rod circumferential rib.

10. The quantitative injection pen according to claim 1, characterized in that, a distal side of the upper housing is further provided with a lower housing buckle; and the lower housing is provided with an upper engagement hole for engaging with the lower housing buckle.

11. The quantitative injection pen according to claim 10, characterized in that, the lower housing buckle has a gradual surface at its distal end and a steep surface at its proximal end.

12. The quantitative injection pen according to claim 1, characterized in that, a distal side of the reset rod is provided with a semi-circumferential rib; and the upper housing is internally provided with a circumferential limiting block for circumferentially engaging with the semi-circumferential rib.

13. The quantitative injection pen according to claim 2, characterized in that, a proximal side of the upper housing is provided with an indicator window; and an inner circumferential surface of the activation sleeve is provided with an identification mark located on the same side as the indicator window.

14. The quantitative injection pen according to claim 1, characterized in that, a proximal side of the push rod is provided with a conical step that tapers towards the distal end; and the reset rod is internally provided with a conical surface that tapers towards the distal end and is configured to engage with the conical step.

15. The quantitative injection pen according to claim 1, characterized in that, the lower housing is internally provided with a cartridge retainer for securing the cartridge; and the cartridge retainer comprises an annular body, a pressing buckle disposed on the annular body for securing the cartridge, and an arched rib disposed on the annular body (701) for abutting against a distal end face of the upper housing.

16. The quantitative injection pen according to claim 10, characterized in that, the cartridge retainer is provided with a hole-position buckle for engaging with the upper engagement hole.

17. The quantitative injection pen according to claim 15, characterized in that, the hole-position buckle has a gradual surface at its distal end and a steep surface at its proximal end.

18. The quantitative injection pen according to claim 15, characterized in that, the lower housing is further provided with a retraction groove to allow the pressing buckle [[(702)]] to move radially outward.

19. The quantitative injection pen according to claim 18, characterized in that, the lower housing is further provided with a lower engagement hole located distally to the upper engagement hole and configured to engage with the hole-position buckle; wherein when the hole-position buckle is engaged with the upper engagement hole, the pressing buckle is located at the same height as the retraction groove, and when the hole-position buckle is engaged with the lower engagement hole, the pressing buckle is disengaged from the retraction groove.

20. The quantitative injection pen according to claim 1, characterized in that, a distal end of the push rod is provided with an engagement step, and a fixed wheel part mounted on the engagement step for urging the piston.

21. The quantitative injection pen according to claim 20, characterized in that, the fixed wheel part comprises a drum-shaped block, a step socket disposed on the drum-shaped block for mounting the engagement step, a rod socket for engaging with a distal end side of the push rod, and a rotation hole disposed at an end of the rod socket and coaxial with the push rod.

22. The quantitative injection pen according to claim 21, characterized in that, the rod socket is radially inwardly contracted.

23. The quantitative injection pen according to claim 21, characterized in that, a distal side of the drum-shaped block is provided with a plurality of friction protrusions for abutting against a proximal end side of the piston.

24. The quantitative injection pen according to claim 1, characterized in that, the lower housing is further provided with an observation window located adjacent to a side of the cartridge.

25. The quantitative injection pen according to claim 1, characterized in that, it further comprises a pen cap; and an outer circumferential surface of the lower housing is further provided with a cap buckle for engaging with the pen cap.

26. The quantitative injection pen according to claim 1, characterized in that, a distal side of the lower housing is further provided with an interface for mounting an injection needle.

27. The quantitative injection pen according to claim 2, characterized in that, a proximal side of the activation sleeve is further provided with an activation button.

28. The quantitative injection pen according to claim 1, characterized in that, the fixed ratchet is arranged around the threaded hole.

29. The quantitative injection pen according to claim 1, characterized in that, the fixed ratchet comprises at least one large ratchet tooth and at least one small ratchet tooth.

30. A method of using the quantitative injection pen according to claim 1, characterized by comprising the following steps:
S01. After an injection needle is inserted into an injection site, rotating the reset rod in a reset rotation direction until it can no longer be rotated;
S02. Rotating the reset rod in an injection rotation direction until it can no longer be rotated.

31. A method of using the quantitative injection pen according to claim 2, characterized by comprising the following steps:
S11. After an injection needle is inserted into an injection site, lifting the activation sleeve in a proximal direction until it can no longer be lifted;
S12. Pressing the activation sleeve in a distal direction until it can no longer be pressed.
